(19) United States
(12) Patent Application Publication
(10) Pub. No.: US 2026/0069434 A1
(43) Pub. Date: Mar. 12, 2026

(54) COMBINED SENSING AND ADJUSTMENT TOOL FOR HIP ARTHROPLASTY BALANCING

(71) Applicant: Zooly Labs, Inc., Nederland, CO (US)

(72) Inventors: Derek Fakhredean Amanatullah, Nederland, CO (US); Moritz Jasper Sharabianlou Korth, Mountain View, CA (US); Michael Thomas LaCour, Knoxville, TN (US)

(21) Appl. No.: 19/325,361

(22) Filed: Sep. 10, 2025

Related U.S. Application Data
(60) Provisional application No. 63/693,135, filed on Sep. 10, 2024.

(51) Int. Cl. A61F 2/46; A61F 2/30; A61F 2/36

(52) U.S. Cl. CPC A61F 2/468; A61F 2/3609; A61F 2/4607; A61F 2002/30492; A61F 2002/365; A61F 2002/4632; A61F 2002/4666

(57) ABSTRACT

A hip balancing sensor system with an integrated ratcheting adjustment mechanism is provided for determining an optimal femoral head offset during hip arthroplasty. The system includes a femoral head offset tool with a head containing a chamber and a collar configured to attach to a femoral stem. The tool comprises force sensors, an IMU, and a spring system configured to automatically adjust femoral head offset by moving the head relative to the collar within a chamber of the head until force equilibrium is achieved. The head is configured to interface with various acetabular structures including acetabular cups, native acetabulum, or acetabular defects. A sensing controller monitors the adjustment process and detects when spring force equals soft tissue resistance indicating optimal positioning. Upon achieving equilibrium, the surgeon measures the femoral head offset distance using a sizing tool and selects a permanent femoral head implant.

### CROSS-REFERENCE TO RELATED APPLICATIONS

This application claims priority to U.S. Provisional Patent Application No. 63/693,135, filed on 10 Sep. 2024, entitled “AUTOMATICALLY ADJUSTING HIP BALANCING TOOL”, the entire specifications of which are incorporated herein by reference.

### BACKGROUND OF THE INVENTION

### Field of the Art

This invention relates generally to the field of arthroplasty and more specifically to a hip balancing sensor system with an integrated ratcheting adjustment mechanism for optimizing soft tissue tensions during hip arthroplasty.

### Discussion of the State of the Art

Hip joint replacement surgery includes the insertion of a prosthetic hip stem into the femur and the attachment of a femoral head to the neck of the hip stem. An acetabular cup is inserted in the acetabulum of the pelvis. The acetabular cup receives the femoral head. However, to provide a stable range of motion for the replacement hip joint, the ligaments of the hip joint need to be adequately tensioned. Optimal soft tissue tension is desired around the hip joint. A hip joint envelope that has too little tension (loose) may result in a small range of motion and may increase the chances of hip dislocation. A hip joint envelope that has too much tension (stiff) may result in a limited range of motion and accelerated implant wear.

Currently, surgeons rely on subjective “feel” and trial-and-error with multiple component sizes to achieve a proper balancing of the hip joint. This approach lacks quantitative measurement, extends surgical time, produces inconsistent results between surgeons, and relies heavily on experience rather than objective data. Further, current computer navigation systems provide position data only, do not integrate with adjustment devices, and require additional setup time. Accordingly, there is a need for an improved system that provides objective measurement of soft tissue tensions while simultaneously enabling automatic adjustment of component positioning to achieve optimal joint balance.

### SUMMARY OF THE INVENTION

Accordingly, the inventor has conceived and reduced to practice, in a preferred embodiment of the invention, a system and method for determining an optimal femoral head offset from a prosthetic hip stem to properly balance a prosthetic hip joint and provide a stable range of motion for the replacement hip joint.

According to a preferred embodiment of the invention, a hip joint balancing system is provided comprising a femoral head offset tool positioned to interface with an acetabular structure. The femoral head offset tool includes a head including a chamber extending linearly from an opening to a closed end. The head is configured to interface with the acetabular structure, and the collar is configured to attach to the femoral stem. The tool further includes force sensors to measure multi-axis forces transmitted through the tool during range of motion testing, an IMU to measure orientation data, and a spring-loaded ratcheting mechanism. The spring-loaded ratcheting mechanism includes a spring system configured to automatically adjust femoral head offset by moving the head relative to the collar by applying spring force against soft tissue resistance. This adjustment happens during surgeon-guided range of motion testing and the adjustment continues until force equilibrium is achieved.

According to another embodiment of the invention, a simplified hip joint balancing tool is provided that includes a head with an internal chamber, a collar for femoral stem attachment, an IMU positioned within the head or collar, a spring-loaded mechanism for automatic adjustment, and a locking mechanism to secure optimal positioning. This simplified embodiment provides automatic femoral head offset adjustment with essential sensing capabilities.

According to a preferred embodiment of the invention, the techniques described herein relate to a femoral head offset tool, including a head including a chamber extending linearly from an opening to a closed-end, a collar linearly movable within the chamber; The mechanism is configured to apply a force between the head and the collar to displace the head relative to the collar from a first position where the collar is proximate the closed end of the chamber to a position spaced away from the closed end, wherein the collar sized to fit over a neck of an implanted prosthetic hip stem, the head is positioned to interface with an acetabular structure, and the displacement of the head to the position is configured to tensions ligaments in the hip capsule to balance a hip joint.

According to an embodiment of the invention, the system further includes a reference module attached to a pelvis to establish orientation reference for measuring relative motion between femur and the pelvis.

According to a preferred embodiment of the invention, sensing controller monitors the automatic adjustment process as the spring force balances against soft tissue resistance, provides real-time feedback during range of motion testing and surgeon manipulation, detect when the spring force equals soft tissue resistance indicating optimal positioning, and provides an indicator that force equilibrium is achieved. The system also includes a display system configured to present real-time feedback during the equilibrium process and surgeon-guided range of motion testing.

According to a preferred embodiment of the invention, the spring-loaded ratcheting mechanism further comprises a spring system calibrated to physiological joint tension requirements, a unidirectional ratchet preventing backward movement of the head relative to the collar, and a locking mechanism to fix position upon achieving optimal balance. The locking mechanism may be selected from the group consisting of: ratchet and pawl system, torsion spring system, pin-based system, and external clamp system.

### BRIEF DESCRIPTION OF THE DRAWING FIGURES

The accompanying drawings illustrate several embodiments of the invention and, together with the description, serve to explain the principles of the invention according to the embodiments. It will be appreciated by one skilled in the art that the particular embodiments illustrated in the drawings are merely exemplary, and are not to be considered as limiting of the scope of the invention or the claims herein in any way.

FIG. 1A is an illustration of a hip balancing sensor system, according to an embodiment of the invention.

FIG. 1B is a schematic view of a prosthetic hip stem with an automatically adjustable femoral head offset tool, according to an embodiment of the invention.

FIG. 2 illustrates a femoral head offset tool with a ratchet locking mechanism, according to an embodiment of the invention.

FIG. 3 illustrates a femoral head offset tool with a torsion spring locking mechanism, according to an embodiment of the invention.

FIG. 4 illustrates a femoral head offset tool with a pin-locking mechanism, according to an embodiment of the invention.

FIG. 5 illustrates a femoral head offset tool with an alternative pin-locking mechanism, according to an embodiment of the invention.

FIG. 6 illustrates a femoral head offset tool with an external clamp locking mechanism, according to an embodiment of the invention.

FIG. 7 illustrates an example system architecture showing the interconnected components and communication pathways of the hip balancing sensor system, according to an embodiment of the invention.

FIG. 8 is a flowchart depicting the complete surgical workflow for using a femoral head offset tool during hip arthroplasty, according to an embodiment of the invention.

FIG. 9 is a flowchart of method depicting sequence of operations performed by the spring-loaded ratcheting mechanism to achieve force equilibrium, according to an embodiment of the invention.

FIG. 10 illustrates a force versus displacement relationship for hip capsule ligaments, demonstrating their non-linear spring properties.

FIG. 11 is a block diagram illustrating an exemplary hardware architecture of a computing device used in an embodiment of the invention.

FIG. 12 is a block diagram illustrating an exemplary logical architecture for a client device, according to an embodiment of the invention.

### DETAILED DESCRIPTION

One or more different inventions may be described in the present application. Further, for one or more of the inventions described herein, numerous alternative embodiments may be described; it should be appreciated that these are presented for illustrative purposes only and are not limiting of the inventions contained herein or the claims presented herein in any way. One or more of the inventions may be widely applicable to numerous embodiments, as may be readily apparent from the disclosure. In general, embodiments are described in sufficient detail to enable those skilled in the art to practice one or more of the inventions, and it should be appreciated that other embodiments may be utilized and that structural, logical, software, electrical, and other changes may be made without departing from the scope of the particular inventions. Accordingly, one skilled in the art will recognize that one or more of the inventions may be practiced with various modifications and alterations. Particular features of one or more of the inventions described herein may be described with reference to one or more particular embodiments or figures that form a part of the present disclosure, and in which are shown, by way of illustration, specific embodiments of one or more of the inventions. It should be appreciated, however, that such features are not limited to usage in the one or more particular embodiments or figures with reference to which they are described. The present disclosure is neither a literal description of all embodiments of one or more of the inventions nor a listing of features of one or more of the inventions that must be present in all embodiments.

Headings of sections provided in this patent application and the title of this patent application are for convenience only and are not to be taken as limiting the disclosure in any way.

A description of an embodiment with several components in communication with each other does not imply that all such components are required. To the contrary, a variety of optional components may be described to illustrate a wide variety of possible embodiments of one or more of the inventions and in order to more fully illustrate one or more aspects of the inventions.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or features of a device may be alternatively embodied by one or more other devices that are not explicitly described as having such functionality or features. Thus, other embodiments of one or more of the inventions need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code that include one or more executable instructions for implementing specific logical functions or steps in the process. Alternate implementations are included within the scope of embodiments of the present invention in which, for example, functions may be executed out of order from that shown or discussed, including substantially concurrently or in reverse order, depending on the functionality involved, as would be understood by those having ordinary skill in the art.

### Definitions

The term “adaptive” as used herein, refers to the capability of a device to automatically adjust its position, orientation, or configuration in response to forces, resistance, or other biomechanical feedback from surrounding tissues without requiring manual intervention.

The term “femoral head offset” as used herein refers to the distance between the center of the femoral head and the axis of the femoral stem along the neck of the hip stem.

The term “acetabular structure” as used herein refers to any anatomical socket or cavity configured to receive a femoral head or prosthetic ball component, including but not limited to an acetabular cup, a native acetabulum, an acetabular defect, a surgically prepared acetabular cavity, or any socket structure in ball-and-socket joint procedures.

The term “acetabular defect” as used herein refers to a surgically created cavity, reamed surface, or prepared socket area in bone tissue, including acetabular defects created during revision procedures, reamed acetabular surfaces prepared for component insertion, or any surgically modified bone socket structure that temporarily or permanently receives a prosthetic ball component.

The term “IMU” or “Inertial Measurement Unit” as used herein refers to an electronic device that measures and reports orientation, angular velocity, and acceleration data using accelerometers, gyroscopes, and magnetometers. The IMU provides real-time spatial positioning information that enables the system to track movement and orientation changes during surgical procedures.

The term “optimal balance” as used herein refers to a state of soft tissue tension around the hip joint that provides adequate stability to prevent dislocation while allowing a full functional range of motion without excess strain on surrounding tissues.

The term “equilibrium point” as used herein, refers to the position where the force applied by the internal mechanism of the femoral head offset tool precisely matches the resistance provided by the hip capsule ligaments and surrounding soft tissues.

The term “biomechanical equilibrium” as used herein refers to a state where opposing forces within the hip joint are balanced to provide optimal stability and function throughout a range of motion.

The terms “spring force equals soft tissue resistance,” “force equilibrium,” and “equilibrium” are used interchangeably throughout this specification to describe the same biomechanical state where the calibrated spring force precisely matches the resistance provided by hip capsule ligaments, indicating optimal positioning for the femoral head offset tool.

The term “spring force” as used herein refers to the calibrated force applied by a spring, magnetic, or other mechanism within the femoral head offset tool, typically ranging from 5-10 Newtons.

The term “hip capsule ligaments” as used herein, refers to the ligamentous structures surrounding the hip joint that provide stability and limit excessive movement, including but not limited to the iliofemoral, pubofemoral, and ischiofemoral ligaments.

The term “locking mechanism” as used herein refers to any device or system that secures the position of the femoral head offset tool components relative to each other, including but not limited to ratchets, pins, clamps, and torsion springs.

The term “hardened behavior” as used herein refers to the non-linear region of ligament stretching where incremental force results in significantly less displacement compared to the linear region, typically occurring beyond 10 mm of displacement for hip capsule ligaments.

The term “automatically adjusting” as used herein refers to a self-directed change in position or configuration that occurs in response to biomechanical forces without requiring manual manipulation by the surgeon.

The term “sizing tool” as used herein refers to a measurement instrument with markings used by the surgeon to measure the precise femoral head offset distance when equilibrium is achieved.

The term “permanent femoral head implant” as used herein refers to the final prosthetic femoral head component selected based on the measured offset distance and installed to replace the femoral head offset tool.

The term “physiological joint tension requirements” as used herein refers to the biomechanical force specifications needed to achieve proper soft tissue balance in the hip joint without excessive strain or inadequate stability.

The term “bone structure” as used herein refers to any anatomical bone or bony landmark suitable for stable attachment of reference sensors, including but not limited to pelvis, scapula, tibia, radius, or other bone structures adjacent to ball-and-socket joints.

The term “physiological joint tension requirements” as used herein refers to the biomechanical force specifications needed to achieve proper soft tissue balance in the hip joint without excessive strain or inadequate stability.

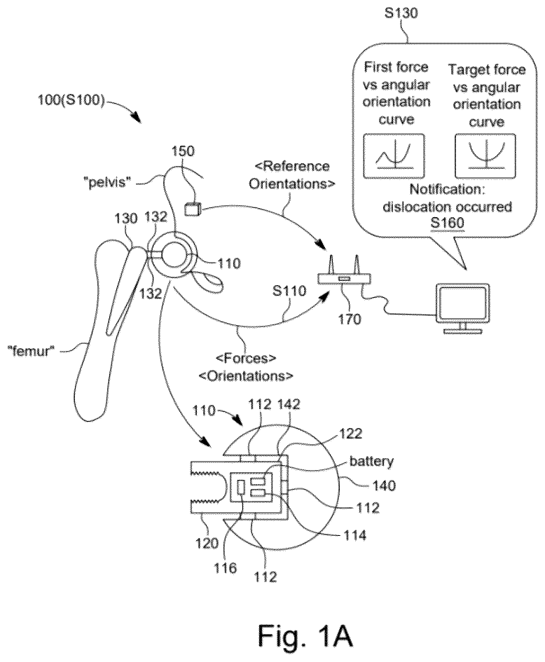

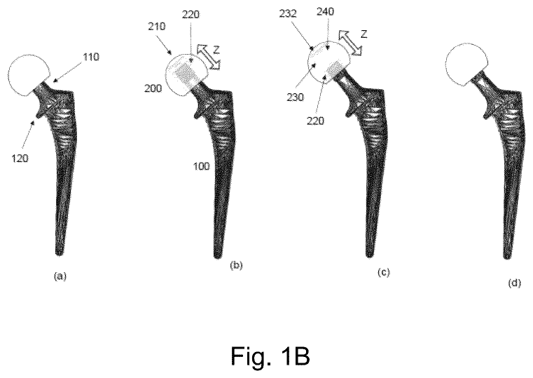

Referring now to Figures, FIG. 1A is an illustration of a hip balancing sensor system, according to an embodiment of the invention.

The primary aim of the hip balancing sensor system is to balance soft tissue tensions around a hip during a partial or total hip arthroplasty. The hip balancing sensor system integrates both measurement capabilities and automatic adjustment in a single device, providing surgeons with objective data to guide component positioning while automatically finding the optimal femoral head offset. This reduces surgical time, improves consistency between surgeons, and leads to better patient outcomes by ensuring optimal soft tissue balance in the hip joint.

In an embodiment, the hip balancing sensor system may include a femoral head offset tool 200, a reference module 150, and a sensing controller 170. In an embodiment, femoral head offset tool 200 may include a shank 120 configured to seat on a proximal end of a neck 132 of a femoral stem component 130 installed on a proximal end of a femur, a spherical shell 140 arranged over shank 120, a set of force sensors 112 coupled to shank 120 and spherical shell 140 and configured to output force data representing forces applied to spherical shell 140, a set of inertial sensors 114 configured to output orientation data representing orientations of spherical shell 140, a spring-loaded ratcheting mechanism (not shown) configured to automatically adjust a position of spherical shell 140 relative to shank 120, and a wireless communication module 116.

In an embodiment, reference module 150 may be configured to couple to a pelvis and includes orientation sensors configured to output reference orientation data representing orientations of the reference module 150. Reference module 150 comprises a clamp assembly configured to attach to pelvic bone structure, a reference IMU housing containing orientation sensors, a wireless transmitter configured to communicate orientation data to sensing controller 170, and a battery compartment containing a rechargeable power source. Reference module 150 is attached to a pelvis to establish orientation reference for measuring relative motion between femur and the pelvis. In particular, reference module 150 may detect changes in position of a patient's pelvis in an inertial reference frame as femoral head offset tool 200 detects changes in position of spherical shell 140 in the inertial reference frame.

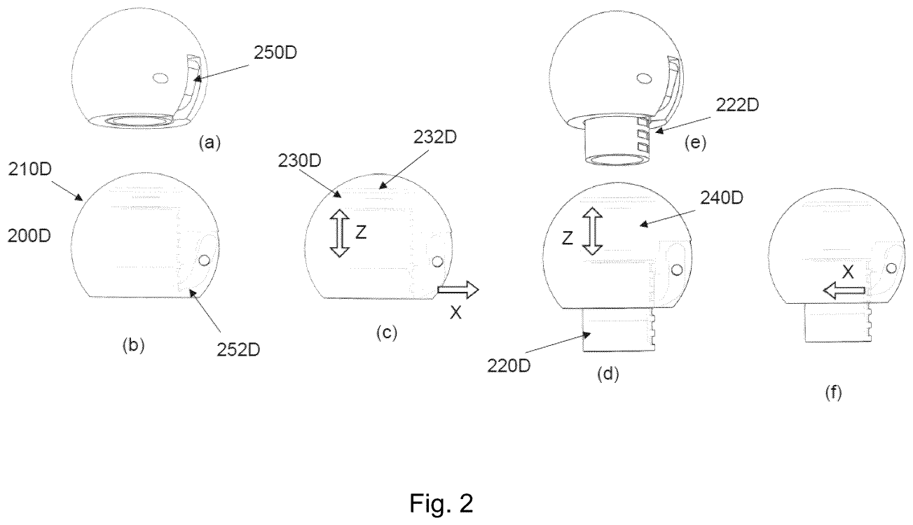

In an embodiment, sensing controller 170 may be configured to access the force data, the orientation data, and the reference orientation data following insertion of automatically adjusting femoral head offset tool 200 into the pelvis during the hip arthroplasty. Sensing controller 170 combines multi-axis force measurements from force sensors 112 with orientation data from IMU 114 to provide superior equilibrium detection. Sensing controller 170 analyzes both data streams simultaneously, detecting when spring force equals soft tissue resistance while confirming positional stability through IMU measurements. This dual-confirmation methodology ensures accurate identification of optimal femoral head positioning by validating force balance through independent position stability verification. More details related to achieving balance and determining femoral head offset are described in FIGS. 2-6 and FIGS. 8-9.

In an embodiment, femoral head offset tool 200 may include a spring-loaded ratcheting mechanism configured to apply a force between components of femoral head offset tool 200. This mechanism allows spherical shell 140 to automatically adjust its position relative to a shank 120 and femoral stem 130 during the range of motion testing. When positioned in the hip capsule, this automated adjustment progressively tensions the ligaments in the hip capsule to achieve optimal balance. The integrated ratcheting mechanism prevents backward movement, ensuring the system only progresses toward improved soft tissue balance. The force sensors 112 and inertial sensors 114 continuously monitor these adjustments, transmitting data via communication module 116 to sensing controller 170, which analyzes the relationship between position and tension to determine the optimal femoral head offset.

In one implementation, shank 120 of femoral head offset tool 200 defines an external support surface 122, and spherical shell 140 defines an internal contact surface 142 facing the external support surface 122 of shank 120. In this implementation, the set of force sensors 112 may be interposed between the external support surface of shank 220 and the internal contact surface of spherical shell 140, locating spherical shell 140 over shank 120 and communicating forces exerted on femoral head offset tool 200 from spherical shell 140 onto shank 120. The spherical shell 140 may seat within an installed acetabular cup as illustrated, engage directly with the patient's native acetabulum before cup installation, or interface with acetabular defects created during surgical preparation or revision procedures. This adaptability allows the system to be deployed at various surgical stages without modification to the core sensing and adjustment mechanisms.

FIG. 1B is a schematic view of a prosthetic hip stem 100 with a femoral head offset tool 200, according to an embodiment of the invention. Femoral head offset tool 200 may be movably coupled to a neck 110 of hip stem 100 in an axial direction Z along neck 110. Femoral head offset tool 200 may include a head 210 and a collar 220 inside head 210 via which femoral head offset tool 200 couples to neck 110. Collar 220 may be movable within chamber 230 (e.g., cylinder) in head 210. The position of femoral head offset tool 200 may be adjustable along neck 110 of hip stem 100 from a negative displaced position relative to a flange 120 or body of hip stem 100 (as shown in FIG. 1B (a) and (b)) and a positive displaced position (as shown in FIG. 1B (b) or (c)).

Head 210 includes a chamber 230 extending linearly from an opening to a closed end. Head 210 is configured to interface with the acetabular cup.

Collar 220 is configured to attach to the femoral stem 110 and remains fixed to the femoral stem during operation. A spring-loaded ratcheting mechanism in the femoral head offset tool 200 has a spring system configured to automatically adjust femoral head offset by moving head 210 relative to collar 220 by applying spring force against soft tissue resistance during surgeon-guided range of motion testing until force equilibrium is achieved.

Collar 220 can move relative to head 210 via a mechanism 240 (linear actuation mechanism) and a lock (not shown) actuatable to lock collar 220 relative to head 210. In one example, mechanism 240 automatically moves collar 220 relative to head 210. In one example, mechanism 240 may be a spring (e.g., coil spring) disposed in chamber 230 between collar 220 and end 232 (closed end) of chamber 230 of head 210, where the spring exerts a force on collar 220 to move collar 220 away from end 232 of chamber 230 and thereby move head 210 away from flange 120 or body of hip stem 100. In another example, mechanism 240 may be one or more magnets (e.g., with opposite polarities) or magnetic material (e.g., metal) that exerts a repulsive force on collar 220 relative to end 232 of chamber 230 to thereby move head 210 away from flange 120 or body of hip stem 100. For example, collar 220 may have a magnet or a magnetic material of a first polarity, and the end 232 of chamber 230 in head 210 can have a magnet or magnetic material of a second polarity (that is the same as the first polarity). In another example, mechanism 240 may be an actuator (e.g., linear actuator, electric motor) that moves collar 220 relative to head 210.

Head 210 may fit into an acetabular cup implanted into an acetabulum of the pelvis of the patient. While FIGS. 1A and 1B describes femoral head offset tool 200 interfacing with acetabular cup, the system is equally configured to interface with a native acetabulum (natural hip socket) or an acetabular defect (surgically prepared cavity or reamed surface). The spherical shell 140 adapts to engage with any of these acetabular structures during different phases of hip arthroplasty procedures.

In various embodiments, head 210 may interface with different types of acetabular structures depending on the surgical procedure and timing. During initial hip arthroplasty procedures, head 210 may be positioned within the native acetabulum before acetabular cup implantation. During revision procedures, head 210 may engage with acetabular defects created by removal of previous components or reaming procedures. In some embodiments, head 210 may interface with surgically prepared acetabular cavities, including reamed surfaces or temporary socket structures. The adaptive mechanism of femoral head offset tool 200 accommodates various acetabular structure geometries and surface conditions, automatically adjusting to achieve optimal soft tissue tension regardless of the specific acetabular structure type.

In another example, head 210 may fit into the acetabulum of the pelvis when an acetabular cup has not been implanted. Head 210 attaches to neck 110 of hip stem 100, which has been previously implanted into the femur of the patient, and extends into the hip envelope or capsule. Mechanism 240 may apply a force between head 210 and collar 220 that moves (e.g., automatically moves) head 210 relative to collar 220 to displace head 210 away from neck 110 and body of hip stem 100, thereby stretching the ligaments of the hip envelope or capsule.

Once the tension of the hip capsule ligaments equates the applied force, femoral head offset tool 200D will be at equilibrium. At this position, the ligaments are properly tensioned without excessive strain. An offset distance of head 210 relative to neck 110 and body of hip stem 100 is measured. Femoral head offset tool 200 would be in its automatically adjusted position at this point, having reached an equilibrium between the hip capsule forces and the internal spring mechanism.

At this point, a locking mechanism (discussed further below) is used to lock the offset position of head 210, and the offset distance is measured or identified to then select a femoral head implant that will provide the measured/identified offset distance. Femoral head offset tool 200 may be detached from hip stem 100 and removed from the hip capsule, and the selected femoral head implant is attached to neck 110 of hip stem 100, and the surgeon can proceed with other steps in the hip replacement surgery.

The sensing controller 170 comprises a computing system with a processor, memory, and specialized software algorithms designed to process and analyze data from the femoral head offset tool 200 and reference module 150. Sensing controller 170 implements a multi-stage data processing pipeline to determine the optimal femoral head offset based on the biomechanical properties of the patient's hip joint.

In an embodiment, sensing controller 170 may include a high-speed microprocessor capable of real-time signal processing, with a dedicated memory for storing sensor data, reference curves, and analysis results. Sensing controller 170 may further include wireless communication modules supporting data transfer from the femoral head offset tool 200 and reference module 150, and a graphical processing unit for rendering force and position visualizations, and input/output interfaces for surgeon interaction and display connection.

In an embodiment, sensing controller 170 may calculate the effective range of motion at different offset positions and identify positions where the range of motion is maximized while maintaining adequate soft tissue tension. Sensing controller 170 provides real-time feedback through graphical visualization, force-displacement curves, 3D force maps across a range of motion, and comparison to target curves displayed on a connected monitor. Further, based on the temporal head offset distance computed by the surgeon, suggestions for optimal femoral head selection may be provided.

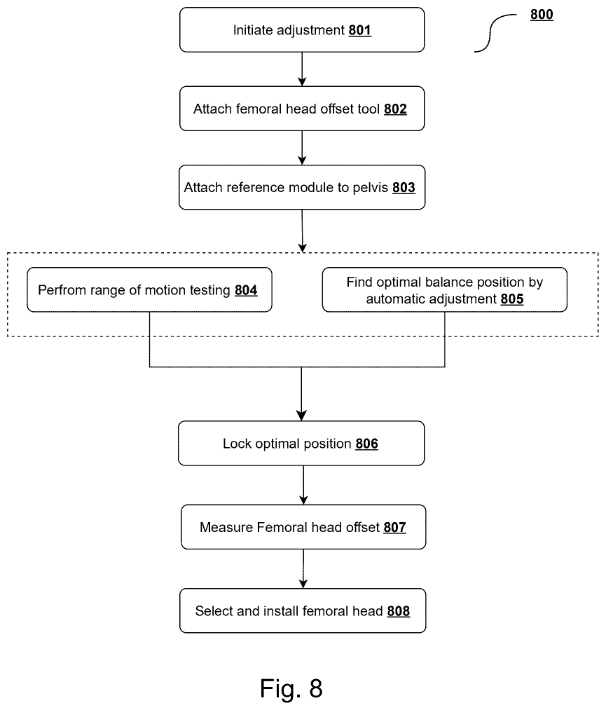

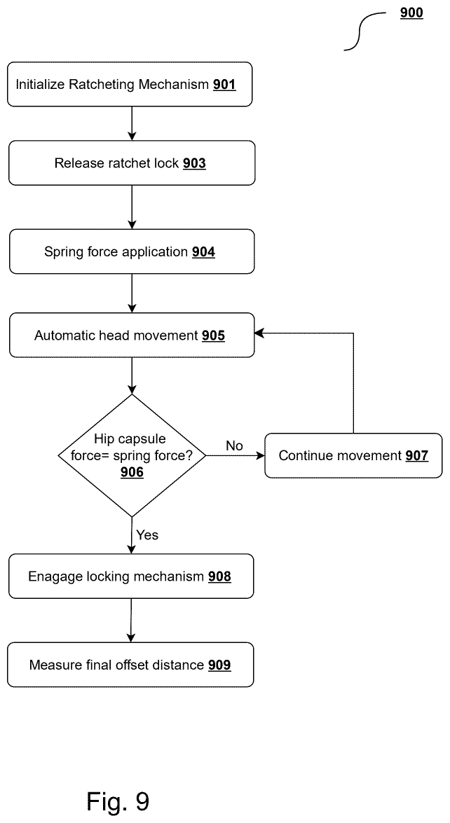

During operation, sensing controller 170 is configured to detect when the spring force equals soft tissue resistance, wherein spring force equaling the soft tissue resistance is indicative of optimal positioning. Responsive to detecting spring force equals soft tissue resistance, sensing controller 170 provides an indicator that force equilibrium is achieved via equilibrium status indicators. The monitoring process further comprises tracking spring force progression in real-time, measuring soft tissue resistance changes, and calculating force equilibrium status during range of motion testing. The real-time feedback during range of motion testing and surgeon manipulation comprises a real-time force visualization interface showing spring force and soft tissue resistance, and equilibrium status indicators to confirm when optimal balance is achieved. Details related to the steps and mechanism are described in FIGS. 8 and 9.

While the present invention is primarily described in the context of hip arthroplasty, the principles and mechanisms disclosed herein are applicable to other ball-and-socket joint procedures, including but not limited to shoulder, knee, elbow, ankle, wrist, and finger joint arthroplasty procedures. The adaptive sensing and adjustment capabilities can be configured for any anatomical socket structure that receives a prosthetic ball component.

FIG. 2 illustrates a femoral head offset tool 200D with a ratchet locking mechanism 240D for determining and adjusting the appropriate femoral head offset during hip replacement surgery. The features of femoral head offset tool 200D are similar to the features of femoral head offset tool 200 in FIG. 1. Thus, the reference numerals used to designate the various components of femoral head offset tool 200D are similar to those used for identifying the corresponding components of femoral head offset tool 200 in FIG. 1, except that a “D” has been added to the reference numeral. Therefore, the structure and description for the various features of femoral head offset tool 200 in FIG. 1 and how it's operated and controlled are understood to also apply to the corresponding features of femoral head offset tool 200D in FIG. 2, except as otherwise described below.

In an embodiment, femoral head offset tool 200D includes a head 210D, a collar 220D, a chamber 230D, a closed end 232D, a mechanism 240D, a pawl 252D, and a teeth recess 222D. Head 210D may be a spherical component that is similar to femoral head offset tool 200 described in FIGS. 1A and 1B and femoral head offset tool 200 interfaces with the acetabulum. Collar 220D may be linearly movable within chamber 230D, designed to fit over neck 110 of an implanted prosthetic hip stem. Chamber 230D is an internal cavity within head 210D that houses collar 220D and mechanism 240D. Closed end 232D is the end of chamber 230D opposite to the opening. A linear actuation mechanism 240D moves collar 220D relative to head 210D.

Femoral head offset tool 200D has head 210D (e.g., spherically shaped head) with a chamber 230D (e.g., cylinder) therein, chamber 230D having an end 232D (closed end). A collar 220D is sized to slidably extend into chamber 230D. Collar 220D (see image 2 (d)) is sized to fit over the end of neck 110 of hip stem 100. Collar 220D may move relative to head 210D via a mechanism 240D (linear actuation mechanism) in chamber 230D, which can be a spring (coil spring), one or more magnets or an actuator (e.g., linear actuator, electric motor), as described above. Femoral head offset tool 200D may include a locking member 250D actuatable to lock and unlock collar 220D relative to head 210D. Pawl 252D is part of the ratchet mechanism that engages with teeth on collar 220D. Teeth/Recesses 222D are present in collar 220D to engage with the pawl. The figure shows multiple images (a-f) depicting the sequential operation of the device.

Images 2(a-f) depict the sequential operation in femoral head offset tool 200D. Image 2(a) shows an initial state with collar 220D fully inserted in head 210D in a retracted position. Image 2(b) displays the internal components of head 210D with pawl 252D engaging the last teeth/recess 222D on collar 220D, locking it in place.

Image 2(c) illustrates the actuation of the ratchet mechanism, pivoting the pawl 252D out of engagement with the teeth 222D, allowing movement along the Z-axis. This corresponds to the “release locking mechanism” and “spring mechanism applies force” steps 903 and 904 described in method 900 (FIG. 9).

Image 2(d) shows collar 220D in an extended position with the mechanism 240D providing force to displace head 210D relative to collar 220D. The spring is calibrated to apply a specific force (between 5-10 Newtons) that tensions the ligaments in the hip capsule.

Image 2(e) depicts the position where equilibrium (the point where the hip capsule ligament forces equal the spring force) is reached. At this position, the ligaments are properly tensioned without excessive strain. Once the tension of the hip capsule ligaments equates to the applied force, femoral head offset tool 200D will be at equilibrium and provide an offset distance of head 210D relative to neck 110 and body of hip stem 100. Femoral head offset tool 200D tool would be in its automatically adjusted position at this point, having reached an equilibrium between the hip capsule forces and the internal spring mechanism. The locking mechanism comprises a locking pin configured to translate through an opening in the head to align with an opening in the collar, and a plurality of teeth. The locking pin engages with one of the teeth to secure the femoral head offset position.

Image 2(f) shows the pawl 252D re-engaged with another tooth/recess 222D of collar 220D in the extended position, locking it in place. This secure locking allows for accurate measurement of the offset distance. Femoral head offset tool 200D may assist surgeons in determining the optimal femoral head offset for balancing hip joint tension.

During hip replacement surgery when the surgeon places the spherical head of the tool into the patient's acetabulum, pawl 252D may be pivoted to disengage from the teeth 222D, allowing the spring mechanism to automatically extend head 210D relative to collar 220D based on the natural resistance of the surrounding tissues. The spring-loaded mechanism automatically adjusts the distance between head 210D and collar 220D based on the resistance provided by the hip joint capsule.

The spring-loaded ratcheting mechanism comprises a calibrated spring system specifically designed to automatically adjust femoral head offset by applying spring force against soft tissue resistance during surgeon-guided range of motion testing until force equilibrium is achieved. The unidirectional ratchet prevents backward movement while allowing progressive adjustment toward optimal positioning.

The system reaches an equilibrium point when the tension in the hip capsule ligaments precisely balances the force applied by the spring mechanism. At this position, the ligaments are properly tensioned without excessive strain. This equilibrium represents the optimal biomechanical state where ligaments are properly tensioned without excessive strain. At this precise point, pawl 252D may be re-engaged with the appropriate tooth/recess 222D to lock the position, capturing the ideal offset measurement. Femoral head offset tool 200D tool would be in its automatically adjusted position at this point, having reached an equilibrium between the hip capsule forces and the internal spring mechanism.

The ratchet mechanism allows for controlled, incremental movement and securely locks the position once optimal tension is achieved. This prevents backward movement while allowing the surgeon to measure the final offset distance, which is used for the selection of the appropriate permanent femoral head implant. This measured offset distance is crucial information that allows the surgeon to select the appropriate permanent femoral head implant that will provide the same offset, ensuring proper ligament tension in the hip joint after surgery.

A sizing tool may be inserted into chamber 230D to measure the precise offset distance between head 210D and collar 220D. The measured offset may be reported to sensing controller 170 that can provide further guidance on implant selection to surgeons. The operation of the ratchet mechanism shown in FIG. 2 follows the workflow illustrated in FIG. 9.

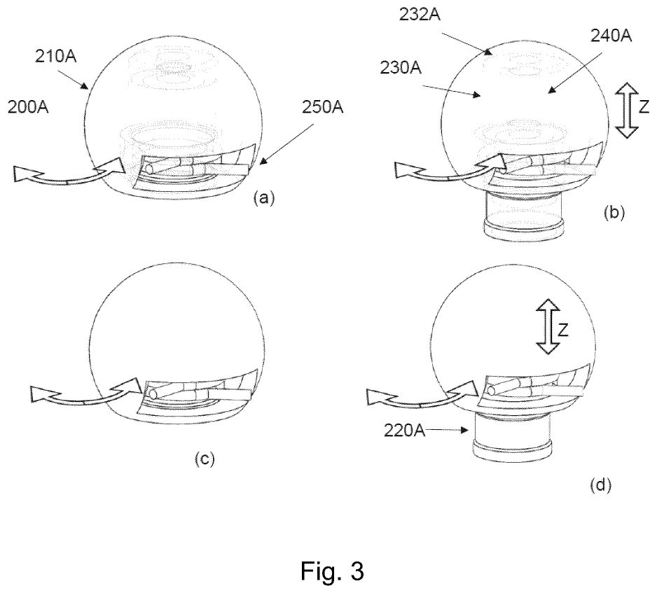

FIGS. 3-6 represents multiple locking mechanisms. FIG. 3 illustrates femoral head offset tool 200A with a torsion spring locking mechanism 240A for determining the appropriate femoral head offset during hip replacement surgery.

The features of femoral head offset tool 200A are similar to the features of femoral head offset tool 200 in FIG. 1. Thus, the reference numerals used to designate the various components of femoral head offset tool 200A are similar to those used for identifying the corresponding components of femoral head offset tool 200 in FIG. 1, except that a “A” has been added to the reference numeral. Therefore, the structure and description for the various features of femoral head offset tool 200 in FIG. 1 and how it's operated and controlled are understood to also apply to the corresponding features of femoral head offset tool 200A in FIG. 3, except as otherwise described below. FIG. 3 shows multiple images (a-d) depicting the sequential operation of the torsion spring locking mechanism 240A.

Images 3(a) and 3(c) show head 210A with semi-transparent rendering to reveal the internal components. Collar 220A may be fully inserted in head 210A in a retracted position, and torsion spring 250A is released (uncompressed), causing its circular opening to contract and engage with the outer surface of collar 220A, securely locking it in place.

Image 3(b) displays the mechanism 240A in transition. When the two arms of the torsion spring 250A are moved toward each other (compressed), the opening of the spring expands, disengaging from collar 220A. This allows head 210A to move axially (in the Z direction) relative to collar 220A. The mechanism 240A provides the force to drive this movement.

Image 3(d) illustrates femoral head offset tool 200 in its extended position. As collar 220A is attached to neck 110 of hip stem 100, mechanism 240A has moved head 210A away from neck 110 of hip stem 100. Releasing the torsion spring will again lock collar 220A relative to head 210A in this extended position. The torsion spring design provides a simple yet effective means to both allow controlled adjustment and secure locking once the optimal position is determined.

During hip replacement surgery when the surgeon places the spherical head of the tool into the patient's acetabulum, the torsion spring is compressed, allowing mechanism 240A to automatically adjust the position of head 210A based on resistance from the hip capsule tissues.

The device finds equilibrium at the position where the hip capsule ligament forces balance with the spring force in the mechanism 240A. This equilibrium represents the optimal biomechanical state where ligaments are properly tensioned. At this equilibrium point, the torsion spring is released to lock the position, which represents the optimal femoral head offset for proper ligament tensioning. This measured offset distance can then be used to select the appropriate permanent femoral head implant that will maintain the same offset, ensuring proper ligament tension in the hip joint after surgery.

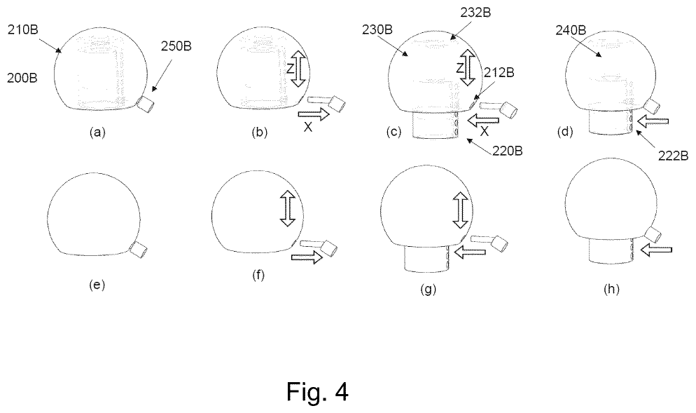

FIG. 4 illustrates a femoral head offset tool 200B with a pin locking mechanism 240B for determining the appropriate femoral head offset during hip replacement surgery. The features of femoral head offset tool 200B are similar to the features of femoral head offset tool 200 in FIG. 1. Thus, the reference numerals used to designate the various components of femoral head offset tool 200B are similar to those used for identifying the corresponding components of femoral head offset tool 200 in FIG. 1, except that a “B” has been added to the reference numeral. Therefore, the structure and description for the various features of femoral head offset tool 200 in FIG. 1 and how it's operated and controlled are understood to also apply to the corresponding features of femoral head offset tool 200D in FIG. 4, except as otherwise described below. FIG. 4 presents eight images (a-h) showing the sequential operation of femoral head offset tool 200B, with the top row images (a-d) displaying semi-transparent views revealing internal components and the bottom row images (c-h) showing external views of the same operational states.

Images in FIG. 4(a) & FIG. 4(c) show collar 220B fully inserted in head 210B in a retracted position with a pin 250B in place, extending through the opening 212B in head 210B and aligned with an opening 222B in collar 220B, locking them together.

Images in FIG. 4(b) & FIG. 4(f) illustrate pin 250B being removed (moved in the X direction), allowing head 210B to move axially (Z direction) relative to collar 220B due to force applied by the mechanism 240B.

Images in FIG. 4(c) & FIG. 4(g) show pin 250B being reinserted through the opening 212B in head 210B and into an opening 222B in collar 220B that has now aligned with head 210B opening at an extended position.

Images in FIG. 4(d) & FIG. 4(h) display the final locked position with collar 220B secured by pin 250B relative to head 210B in the extended position.

This pin-locking design allows for discrete positioning of femoral head offset tool 200. During hip replacement surgery when the surgeon places the spherical head of the tool into the patient's acetabulum, pin 250B is removed, allowing mechanism 240B to automatically adjust the position of head 210B based on resistance from the hip capsule tissues.

The system reaches an equilibrium point when the tension in the hip capsule ligaments balances with the force applied by mechanism 240B. This equilibrium represents the optimal biomechanical state where ligaments are properly tensioned. At this point, the surgeon can insert the pin through the aligned openings to lock the position.

The multiple openings 222B in collar 220B allow for precise locking at various discrete positions, capturing the specific offset that provides optimal tension. This measured offset can then be used to select the appropriate permanent femoral head implant.

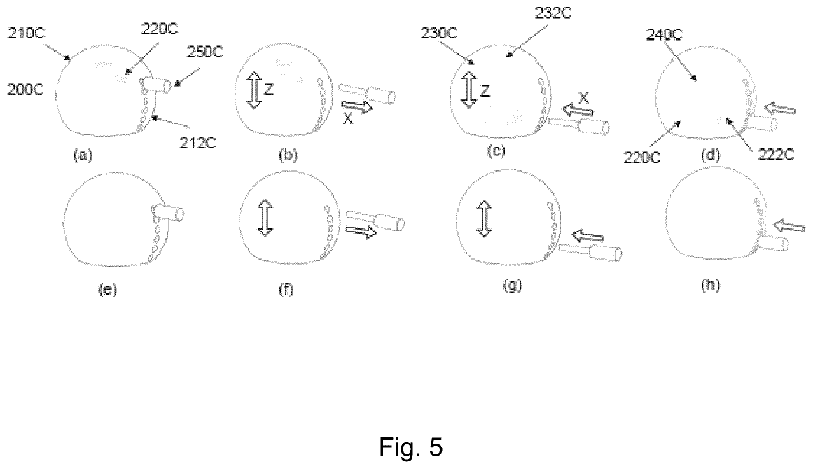

FIG. 5 illustrates a femoral head offset tool 200C with an alternative pin locking mechanism 240C for determining the appropriate femoral head offset during hip replacement surgery. The features of femoral head offset tool 200C are similar to the features of femoral head offset tool 200 in FIG. 1. Thus, the reference numerals used to designate the various components of femoral head offset tool 200C are similar to those used for identifying the corresponding components of femoral head offset tool 200 in FIG. 1, except that a “B” has been added to the reference numeral. Therefore, the structure and description for the various features of femoral head offset tool 200 in FIG. 1 and how it's operated and controlled are understood to also apply to the corresponding features of femoral head offset tool 200C in FIG. 5, except as otherwise described below. FIG. 5 presents eight images (a-h) showing the sequential operation of femoral head offset tool 200C.

Images in FIG. 5(a) & FIG. 5(e) show collar 220C fully inserted in head 210C in a retracted position with the pin 250C extending through one of the openings 212C in head 210C and the opening 222C in collar 220C, locking them together.

Images in FIG. 5(b) & FIG. 5(f) illustrate pin 250C being removed (moved in the X direction), allowing head 210C to move axially (Z direction) relative to collar 220C.

Images in FIG. 5(c) & FIG. 5(g) show pin 250C being reinserted through a different opening 212C in head 210C and opening 222C in collar 220C that is now aligned with that head opening.

Images in FIG. 5(d) & FIG. 5(h) display the final locked position with collar 220C secured by the pin 250C relative to head 210C in the extended position.

The multiple openings in head 210C (instead of collar 220C) allow for discrete positioning of the femoral head offset.

The system reaches an equilibrium point when the tension in the hip capsule ligaments balances with the force applied by mechanism 240C. This equilibrium represents the optimal biomechanical state where ligaments are properly tensioned. At this point, the surgeon can insert the pin through the appropriate head opening that aligns with collar 220C opening to lock the position.

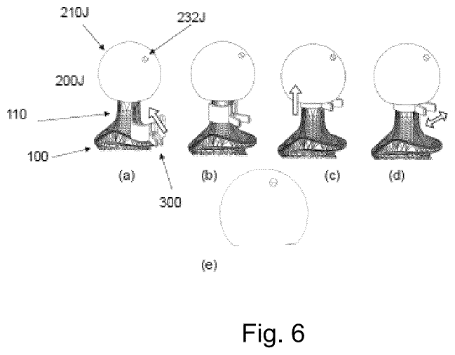

FIG. 6 illustrates a femoral head offset tool 200J with an external clamp locking mechanism 240J, showing how the tool interfaces with hip stem 100 implant and the process of securing the optimal offset position. The features of femoral head offset tool 200J are similar to the features of femoral head offset tool 200 in FIG. 1. Thus, the reference numerals used to designate the various components of femoral head offset tool 200J are similar to those used for identifying the corresponding components of femoral head offset tool 200 in FIG. 1, except that a “J” has been added to the reference numeral. Therefore, the structure and description for the various features of femoral head offset tool 200 in FIG. 1 and how it's operated and controlled are understood to also apply to the corresponding features of femoral head offset tool 200J in FIG. 6, except as otherwise described below. FIG. 6 presents five images (a-e) showing the sequential operation of femoral head offset tool 200J.

Image in FIG. 6(a) shows femoral head offset tool 200J attached to neck 110 of hip stem 100, with a clamp 300 positioned nearby but not yet engaged. Clamp 300 may be an external spring-loaded clamp that locks the position of head 210J relative to neck 110.

Image in FIG. 6(b) illustrates femoral head offset tool 200J in position on neck 110 of hip stem 100, prior to the clamp engagement. Femoral head offset tool 200J would be in its automatically adjusted position at this point, having reached an equilibrium between the hip capsule forces and the internal spring mechanism.

Image in FIG. 6(c) shows the clamp 300 being compressed (opened) to prepare for placement around neck 110.

Image in FIG. 6(d) depicts clamp 300 positioned around neck 110 and released, securing it in place. Clamp 300 may then be slid upward into head 210J.

Image in FIG. 6(e) shows a different view of the assembly, highlighting how clamp 300 fits between neck 110 and the wall of chamber 232J to prevent further movement of collar 220J relative to head 210J.

This clamp-based locking mechanism provides an alternate approach to securing the femoral head offset position. Once the femoral head offset tool 200J has automatically adjusted to the optimal position where hip capsule ligament tension balances the internal spring force, the surgeon uses the clamp 300 to lock this position.

The clamp is first compressed to open it, then placed around neck 110 of hip stem 100. When released, clamp 300 closes securely around neck 110. The surgeon then slides clamp 300 upward into the space between neck 110 and the inner wall of chamber 232J. This effectively prevents any further movement of collar 220J relative to head 210J, locking in the optimal offset position. This approach inhibits further movement while allowing the surgeon to measure the precise offset distance using a sizing tool. The measured offset then guides the selection of the permanent femoral head implant that will provide the same optimal offset, ensuring proper ligament tension in the hip joint after surgery.

The clamp-based design offers the advantage of a secure external lock that can be applied after finding the equilibrium position, without requiring pre-positioned locking features like pins or ratchets. This may provide additional flexibility in achieving the precise offset needed for optimal hip balance.

### Simplified Hip Joint Balancing Tool

In an alternative embodiment, a simplified hip joint balancing tool provides automatic femoral head offset adjustment without requiring external sensing controllers, display interfaces, or wireless communication systems. This embodiment focuses on the core mechanical adjustment and equilibrium detection capabilities while maintaining cost-effectiveness and ease of use.

The simplified tool includes head 210 with internal chamber 230, collar 220 for femoral stem attachment, spring-loaded mechanism 240, locking mechanism, and a single IMU positioned within either head 210 or collar 220. Unlike the full system embodiment, this simplified version operates independently without sensing controller 170, reference module 150, or display interface 716.

During operation, the surgeon relies on tactile feedback, visual observation, and professional judgment to detect when the spring mechanism reaches equilibrium with hip capsule resistance. The IMU provides orientation feedback that can be accessed through simple indicator lights, audible signals, or basic readout display integrated within the tool itself, eliminating the need for external controllers or complex user interfaces.

This simplified approach offers several advantages including, but not limited to, reduced manufacturing complexity and cost, faster regulatory approval due to fewer electronic components, enhanced reliability with fewer failure points, simplified sterilization procedures, reduced training requirements for surgical staff, and broader market accessibility, particularly in cost-conscious healthcare environments. The integrated IMU provides essential feedback for confirming optimal positioning while maintaining the system's simplicity and reliability.

The automatic adjustment mechanism operates identically to the full system embodiment—the spring applies calibrated force until soft tissue resistance creates equilibrium. However, equilibrium detection relies on the surgeon's clinical assessment of joint stability and range of motion rather than algorithmic analysis. The surgeon observes when further spring extension produces minimal additional joint lengthening, indicating optimal soft tissue tension has been achieved.

This simplified embodiment maintains the core innovation of automatic, force-guided femoral head offset adjustment while providing a practical solution for healthcare providers who prioritize mechanical reliability and cost-effectiveness over comprehensive electronic monitoring capabilities.

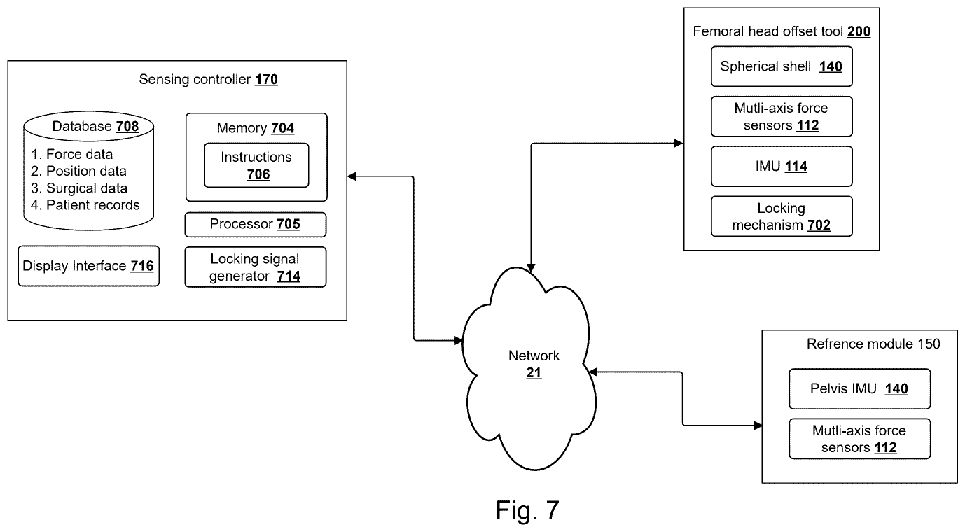

FIG. 7 illustrates an example hip balancing sensor system 700 architecture showing the interconnected components and communication pathways of the hip balancing sensor system, according to an embodiment of the invention.

Hip balancing sensor system 700 operates through a comprehensive wireless communication network that enables real-time data exchange and coordinated control between all system components. Network 21 establishes secure, low-latency communication channels femoral head offset tool 200, reference module 150, and sensing controller 170 through wireless protocols optimized for surgical environments.

Sensing controller 170 serves as the central processing, control, and user interface system, comprising multiple functional modules organized for comprehensive surgical support.

Database 708 may store and manage multiple data types including force measurements from multi-axis force sensors 112, position and orientation data from IMU 114 and pelvis IMU, surgical procedure timestamps and parameters, and patient-specific records for post-operative analysis. Database 708 may store integrated analysis results combining force and position data, correlation patterns between force measurements and spatial positioning, and equilibrium validation records confirming both force balance and positional stability.

Processor 505 may execute instructions 706 in real-time to analyze sensor data from both femoral head offset tool 200 and reference module 150, calculate relative motion between femur and pelvis, and process raw sensor data into meaningful information for surgical guidance. Processor 505 performs integrated analysis by combining multi-axis force measurements with IMU orientation data to achieve superior equilibrium detection accuracy. Processor 505 analyzes force and IMU data streams in parallel, tracking both force progression and position changes as the spring-loaded ratcheting mechanism adjusts the femoral head offset. Force equilibrium detection involves monitoring multi-axis force measurements for patterns indicating that spring force has balanced against soft tissue resistance, while positional stability detection processes IMU orientation data to identify when automatic adjustment movement has ceased.

Display interface 716 presents comprehensive surgical information and control options to the surgical team, including manual lock control interface for surgeon-controlled engagement of locking mechanism 702.

Femoral head offset tool 200 is a sensing unit and represents the primary measurement and adjustment device positioned between the femoral stem and acetabular cup. Femoral head offset tool 200 comprises spherical shell 140 configured to interface with the acetabular cup and provide the primary contact surface for force transmission during range of motion testing. Multi-axis force sensors 112 may be arranged within spherical shell 140 to measure forces in x, y, and z axes during hip joint movement. IMU 114 (inertial measurement unit) is configured to measure orientation, angular velocity, and acceleration of the femoral component relative to anatomical reference frames. A spring-loaded ratcheting mechanism may be used by surgeons to automatically adjust femoral head offset by balancing calibrated spring force against soft tissue resistance until equilibrium is reached.

Locking mechanism 702 automatically engages when the spring mechanism reaches force equilibrium, securing the optimal femoral head offset position and preventing further adjustment

Locking mechanism 702 in femoral head offset tool 200 may be configured to be manually engaged by the surgeon to secure the femoral head offset position, preventing further adjustment after the surgeon confirms proper joint balance

Reference module 150 is configured for attachment to the pelvic structure and includes pelvis IMU 140 and multi-axis force sensors 112. Pelvis IMU 140 may measure pelvic orientation and movement, providing the reference frame for calculating relative motion between femur and pelvis. Multi-axis force sensors 112 are positioned to measure reference forces and provide baseline measurements for comparative analysis with femoral head offset tool 200.

During operation, femoral head offset tool 200 continuously transmits multi-axis force measurements from multi-axis force sensors 112 and orientation data from IMU 114 to sensing controller 170 via network 21. Simultaneously, reference module 150 transmits pelvic orientation data from pelvis IMU 140 and reference force measurements, enabling processor 705 to calculate relative motion and joint positioning in real-time. Sensing controller 170 processes this incoming data stream using instructions 706 stored in memory 704 and executed by processor 505, converting raw sensor data into meaningful force distributions, balance status indicators, and positioning information for surgical guidance. The integrated analysis combines independent force and position assessments to generate comprehensive equilibrium determination. Force sensors may indicate force stabilization while the mechanism is still micro-adjusting, or position sensors may detect temporary stability during force transitions. Only when both force balance and positional stability criteria are simultaneously satisfied does the sensing controller 170 confirm that true equilibrium has been achieved

The spring-loaded ratcheting mechanism within femoral head offset tool 200 automatically adjusts femoral head offset based on measured forces exceeding the calibrated spring resistance thresholds.

Display interface 716 presents real-time feedback to the surgical team, enabling the surgeon to assess joint balance and manually engage locking mechanism 702 when optimal positioning is determined. Throughout the procedure, database 708 records all measurements, adjustment actions, and surgeon inputs for surgical documentation and post-operative analysis. Display interface 716 displays force progression graphs showing multi-axis force measurements over time, position stability indicators derived from IMU data, and integrated equilibrium status combining both data sources. During surgeon-guided range of motion testing, display shows live force distributions across flexion-extension, abduction-adduction, and internal-external rotation movements while simultaneously presenting position tracking graphics that illustrate spatial movement of the femoral head offset tool. A visual confirmation may be generated when spring force equals soft tissue resistance and positional stability is achieved simultaneously.

In addition to the above the display interface 716 may include indicators of battery levels, wireless connectivity, and sensor functionality. Trend graphs showing force progression and balance convergence over time may also be part of the display interface. This real-time force visualization, indicators and alerts assist surgeon decision-making. The equilibrium detection interface provides clear visual confirmation when both force balance and positional stability have been achieved, with force equilibrium indicators showing when spring force matches soft tissue resistance across all measured axes, and position stability indicators confirming when IMU data shows cessation of adjustment movement.

In some cases, a range of motion graphics dashboard displays graphical representations of hip joint movement, show relative positioning between femur and pelvis based on data from IMU 114 and pelvis IMU 140, and provide visual guidance for range of motion testing procedures. This dual-stream visualization enables surgeons to observe the correlation between applied forces and resulting positional adjustments, providing comprehensive joint analysis during range of motion testing.

The simplified interface design enables the surgeon to monitor the automatic equilibrium process through the real-time display showing force balance progression. This streamlined approach combines mechanical automation with surgical oversight, where the spring-loaded mechanism automatically finds its equilibrium point against soft tissue resistance while providing the surgeon with essential monitoring and documentation capabilities through two distinct, purpose-focused interface areas.

While the detailed embodiments described above focus specifically on hip arthroplasty applications, the fundamental principles of the spring-loaded ratcheting mechanism, force sensing, and automatic adjustment capabilities are readily adaptable to other ball-and-socket joint procedures. The femoral head offset tool concept can be scaled and configured for shoulder arthroplasty (with humeral head components), as well as other spherical joint interfaces in knee, elbow, ankle, wrist, and finger joint procedures.

For shoulder applications, the tool would interface with glenoid socket structures (natural glenoid, glenoid defects, or glenoid components) while attached to humeral stem components. The spring calibration, force sensing ranges, and locking mechanisms can be adjusted for the different biomechanical requirements of shoulder joint soft tissues. Similarly, smaller-scale versions could be developed for finger joint arthroplasty procedures, with appropriately scaled force ranges and component dimensions.

The sensing controller 170 and reference module 150 concepts apply universally to ball-and-socket joint procedures, with reference modules attachable to appropriate bone structures (scapula for shoulder procedures, adjacent bone structures for other joints) to establish orientation references for relative motion measurement.

FIG. 8 is a flowchart depicting the complete surgical workflow for using femoral head offset tool 200 during hip arthroplasty and provides a sequential step-by-step process from initial setup through the selection of the permanent femoral head implant.

At step 801, adjustment is initiated by the surgeon. The initiation may include the resection of the proximal end of the patient's femur, preparation of femoral head offset tool 200, and confirmation that the femoral head offset tool's locking mechanism is engaged in the retracted position. Femoral head offset tool 200 is powered on, and proper functionality is verified (battery level, sensors, etc.) Further, sensing controller 170 and display are set up in the operating field. This preparation ensures the tool is ready for accurate measurement and is properly configured before attachment to the femoral stem.

At step 802, a femoral head offset tool 200 is attached by the surgeon. The surgeon may select an instrument femoral head offset tool of appropriate size that approximates the patient's native femoral head. The surgeon carefully attaches femoral head offset tool 200 to the proximal end of the femoral stem 100 component that has been installed in the femur. The surgeon ensures collar 220 of the tool fits securely over neck 110 of the femoral stem.

At step 803, reference module 150 to the pelvis is attached by the surgeon. The surgeon identifies an accessible, stable region of the patient's pelvis, and using an adhesive, threaded fastener, or pin system, the surgeon secures reference module 150 to the pelvis. The surgeon verifies that reference module 150 is firmly attached and will not shift during hip manipulation. Further, the surgeon confirms wireless communication between the reference module 150 and sensing controller 170. Reference module 150 tracks the pelvis position in the inertial reference frame, allowing the system to calculate the relative motion between the femur and pelvis during testing.

At step 804, the surgeon may perform a range of motion testing. The surgeon may seat femoral head offset tool 200 into the patient's acetabulum. The surgeon indicates a neutral position of the hip through sensing controller 170 (via direct input, voice command, or foot pedal) and methodically moves the hip through its range of motion, which may include: flexion-extension motion, abduction-adduction motion, and internal-external rotation. Motion mapping allows the system to collect reference data on the hip's range of motion and the corresponding forces at different positions. During range of motion testing, sensing controller 170 continuously processes integrated force and IMU data, analyzing multi-axis forces while tracking spatial positioning to build comprehensive joint movement profiles. The integrated analysis enables real-time assessment of force distributions across different movement patterns while confirming positional stability throughout the range of motion cycle.

Performing range of motion testing may include moving the hip through flexion-extension motion, abduction-adduction motion, and internal-external rotation. During this testing, the system measures multi-axis forces between the femoral stem and the acetabular cup while automatically adjusting femoral head offset by applying spring force against soft tissue resistance until force equilibrium is achieved.

The spring system is calibrated to physiological joint tension requirements and applies force between 5-10 Newtons. The spring force is specifically calibrated to position ligaments at a transition point between linear behavior and hardened behavior, ensuring optimal tension without excessive strain. This calibration ensures that the automatically adjusting femoral head offset achieves optimal hip joint tension during the range of motion testing.

Sensing controller 170 monitors the automatic adjustment process as the spring force balances against soft tissue resistance and provides real-time feedback during range of motion testing and surgeon manipulation.

At step 805, the surgeon finds an optimal balance position by automatic adjustment. The surgeon disengages the locking mechanism of femoral head offset tool 200 (depending on the mechanism type 240A, 240B, 240C, 240D and 240J) and the mechanism moves head 210 relative to collar 220 until equilibrium is reached. The surgeon may gently manipulate the hip joint to ensure true equilibrium is found. When femoral head offset tool 200 automatically finds the precise position where soft tissue tension balances the spring force, representing the optimal offset for proper ligament tensioning.

An automatic adjustment is performed that progressively moving head 210 away from collar 220 automatically to adjust femoral head offset by applying spring force against soft tissue resistance until force equilibrium is achieved. Through this adjustment collar 220 remains fixed to femoral stem 110. This movement continues until the spring force balances against soft tissue resistance, with the unidirectional ratchet preventing backward movement of head 210 relative to collar 220.

At step 806, the surgeon locks the optimal position in femoral head offset tool 200. Once equilibrium is achieved, the surgeon engages the locking mechanism. For torsion spring mechanism 240A, the surgeon may release the spring arms to contract around collar 220A. For ratchet mechanism 240D, the torsion spring allows pawl 252D to engage with the appropriate tooth 222D. For pin-based locking mechanisms 240B, the surgeon may reinsert the pin 250B through aligned openings. For clamp locking mechanism 240J, the surgeon may apply an external clamp 300. Locking preserves the exact offset position determined by the equilibrium between soft tissue tension and spring force, ensuring accurate measurement.

At step 807, the surgeon uses a sizing tool with markings to measure the precise offset. The sizing tool may be inserted into chamber 230 between the wall and neck 110 of the hip stem 100. The surgeon may the marking visible at the appropriate reference point. In an embodiment, the measurement may be automatically recorded by sensing controller 170. This quantitative measurement will directly inform the selection of the permanent femoral head implant that will maintain the same offset.

At step 808, the surgeon may select and install femoral head 210. Based on the measured offset, the surgeon may select a permanent femoral head implant of appropriate size and offset. Femoral head offset tool 200 is removed from the femoral stem, and the selected permanent femoral head implant is installed on the femoral stem 100. This selection process ensures that the final implant maintains the optimal positioning determined through the automatic adjustment and equilibrium detection process.

Unlike existing systems that require separate measurement and adjustment tools, the present invention uniquely integrates both functions in a single device with automatic real-time adjustment based on biomechanical feedback.

FIG. 9 is a flowchart of method 900 depicting sequence of operations performed by the spring-loaded ratcheting mechanism to achieve force equilibrium. Method 900 describes the process of adjusting the offset distance between head 210 and collar 220 by moving the head relative to the collar based on the resistance provided by the hip joint capsule. This adjustment effectively changes the offset distance (the distance between the femoral stem and the center of the femoral head).

At step 901, the ratcheting mechanism is initialized in the retracted position. Head 210 is positioned in its closest position relative to collar 220, and collar 220 is configured to attach to the femoral stem 110. Head 210 is fully retracted relative to collar 220. Method 900 is initiated when the locking mechanism is engaged to maintain this retracted position before automatic adjustment begins.

For torsion spring mechanism 240A, the spring is in a contracted position around collar 220A. For ratchet mechanism 240D, pawl 252D to engage with the innermost tooth 222D. For pin-based locking mechanisms 240B, pin 250B is inserted through aligned openings at the innermost position. This initial configuration ensures head 210 starts in its closest position to the femoral stem 100, allowing the mechanism to extend outward to find the optimal position.

At step 903, after the tool is attached to the femoral stem and seated in the acetabulum, the surgeon disengages or releases the specific locking mechanism. In the case of a torsion spring mechanism 240A, the surgeon may compress the arms of the spring to expand its opening. In the case of a pin locking mechanism 240B, the surgeon may remove pin 250B by moving it laterally. In the case of a ratchet locking mechanism 240D, the surgeon pivots pawl 252D away from the teeth/recesses 222D. In the case of clamp locking mechanism 240J, the surgeon may ensure that the clamp is not yet applied. The unlocking operation allows the mechanism to freely adjust based on the balance between spring force and soft tissue resistance.

At step 904, the spring mechanism in femoral head offset tool 200 automatically applies force between head 210 and collar 220. The spring mechanism creates a consistent, calibrated force that acts between head 210 and collar 220 components. The spring force is calibrated to position ligaments at a transition point between linear behavior and hardened behavior.

The spring mechanism may include a coil spring arranged in chamber 230 between collar 220 and a closed end, magnets with opposing polarities, an electromagnetic actuator, or a compressed gas mechanism. This consistent, calibrated force application is what drives the automatic adjustment process, eliminating subjective assessment by the surgeon.

At step 905, head 210 interfaces with the acctabulum, and applied force drives head 210 away from collar 220 until it reaches a force equilibrium where the resistance from the hip capsule tissues matches the spring force.

The automatic adjustment comprises progressively moving head 210 away from collar 220 automatically to adjust femoral head offset by applying spring force against soft tissue resistance until force equilibrium is achieved. Head 210 moves automatically to adjust femoral head offset by applying spring force against soft tissue resistance during surgeon-guided range of motion testing until equilibrium is achieved. The spring-loaded mechanism causes head 210 to move unidirectionally away from collar 220, which remains fixed to the femoral stem neck 110, thereby increasing the offset distance between the head and the stem.

The force from the spring mechanism causes head 210 to move axially away from collar 220. As collar 220 is fixed to the femoral stem, this effectively increases the distance between head 210 and stem 100. This movement continues until resistance from the hip capsule tissues balances the spring force. The movement stops when the hip capsule ligaments begin to exhibit hardened behavior.

This adaptive equilibrium-seeking process represents the fundamental innovation of the femoral head offset tool 200. As the surgeon manipulates the hip through its range of motion, the tool continuously adapts to changing force vectors and tissue resistances, seeking the optimal balance point automatically. This adaptation occurs in real-time and responds to patient-specific tissue characteristics rather than relying on predetermined settings. The adaptive nature of this process accommodates variations in patient anatomy, tissue quality, and surgical approach, making it superior to static measurement or trial-and-error techniques. By adapting to the specific biomechanical profile of each patient's hip joint, the tool ensures that component selection will provide optimal function for that individual's unique anatomical and physiological conditions.

At step 906, femoral head offset tool 200 detects when spring force equals soft tissue resistance, wherein spring force equaling the soft tissue resistance is indicative of optimal positioning. The system reaches a natural force equilibrium point where resistance from the hip capsule tissues exactly balances the spring force. This force equilibrium represents the biomechanically optimal point where ligaments are properly tensioned without excessive strain.

The determination of force equilibrium involves integrated analysis of both force sensor data and IMU orientation data. Sensing controller 170 processes multi-axis force measurements to detect force balance patterns while simultaneously analyzing IMU data to confirm positional stability. True equilibrium is confirmed only when both force balance and position stability criteria are satisfied, providing dual validation of optimal positioning.

If force equilibrium is not yet reached, then at step 907, the method 900 continues with further movement. The detection process involves monitoring force progression in real-time and identifying when incremental force results in minimal displacement, confirming biomechanical equilibrium across multiple axes. This is the critical force equilibrium finding step where the device automatically determines the optimal femoral head offset based on the patient's individual anatomy.

The system detects when spring force equals soft tissue resistance, wherein spring force equals the soft tissue resistance is indicative of optimal positioning. This detection process involves monitoring force progression in real-time and identifying when incremental force results in minimal displacement, confirming biomechanical equilibrium across multiple axes. The achievement of force equilibrium is determined by analyzing force symmetry across multiple axes during a complete range of motion cycle, ensuring that the equilibrium represents true biomechanical balance rather than temporary resistance.

If equilibrium is reached, then at step 908, the surgeon engages the appropriate locking mechanism. The locking mechanism is configured to fix position upon achieving optimal balance and may be selected from the group consisting of: ratchet and pawl system, torsion spring system, pin-based system, and external clamp system. The locking mechanism secures the femoral head offset position upon achieving optimal balance, preventing any backward movement of head 210 relative to collar 220 using a unidirectional ratchet mechanism that prevents backward movement of head 210 relative to collar 220.

In the case of torsion spring locking mechanism 250A, the surgeon releases the spring arms to contract onto collar 220A. In the case of ratchet locking mechanism 240D, the surgeon allows pawl 252D to engage with the appropriate tooth 222D. In the case of pin-based locking mechanism 240B, the surgeon reinserts pin 250B through the now-aligned openings. In the case of the clamp locking mechanism, the surgeon places and secures clamp 300 around neck 110. This locking step preserves the exact position determined by the biomechanical equilibrium, ensuring that the measurement taken will reflect the optimal offset.

The locking mechanism engagement prevents backward movement of head 210 relative to collar 220 the spring-loaded collar 220, securing the securing the head at the optimal femoral head offset position upon achieving optimal balance. This prevents any inadvertent changes to the precisely determined offset distance.

At step 909, the surgeon measures the offset distance the distance between head 210 and collar 220 based on the resistance provided by the hip joint capsule. The surgeon measures, using a sizing tool, the femoral head offset distance when equilibrium is achieved. The sizing tool provides precise measurement capabilities with markings that indicate the exact offset distance. This measurement is critical for selecting a permanent femoral head implant based on the measured offset distance, ensuring that the final implant will maintain the optimal positioning determined during the equilibrium process. The markings on the sizing tool indicate the precise offset distance. Further, the measurement may be digitally recorded or displayed.

The novel combination of real-time force sensing with automatic mechanical adjustment in a single integrated device represents a significant advancement over prior art systems that require separate measurement tools and manual trial-and-error component selection

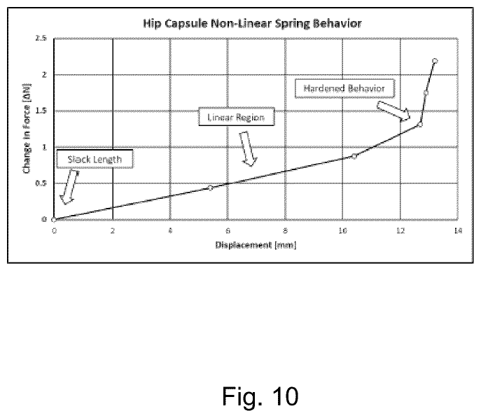

FIG. 10 illustrates a force versus displacement relationship for hip capsule ligaments, demonstrating their non-linear spring properties.

The adaptive mechanism of femoral head offset tool 200 is specifically designed to respond to the complex non-linear properties of hip joint soft tissues. This adaptive response is critical because hip capsule ligaments exhibit variable stiffness across their range of motion, requiring a sensing and adjustment system that can accommodate these changing properties. The tool's adaptive nature is particularly valuable in finding the transition point between the linear region and hardened behavior of ligaments, which represents the optimal tensioning point for long-term joint stability and function. By automatically adapting to these tissue-specific characteristics, the tool achieves personalized component positioning that would be extremely difficult to determine through manual testing alone.

The graph in FIG. 10 represents experimental data collected using femoral head offset tool 200. This graph demonstrates that hip capsule ligaments display non-linear spring properties characteristic of a hardening spring. This was determined experimentally using the femoral head offset tool.

In the graph, after the slack length is reached, changes in force induce large amounts of ligament stretch (displacement) in the linear region. The ligaments eventually reach a non-linear region where they display hardened behavior, where changes in force induce significantly less stretch compared to the linear region. This transition point between linear behavior and hardened behavior represents a critical threshold for ligament tensioning. An initial region (0-2 mm) is called slack length is where minimal force results in some displacement. The middle section (approximately 2-10 mm) represents a liner region where force and displacement have a roughly proportional relationship. Final region (beyond 10 mm) shows a steep increase in force with minimal additional displacement indicative of hardened behavior.

The force applied by the mechanism (spring, magnets, linear actuator, etc.) in femoral head offset tool 200 is specifically calibrated based on this data. According to the specification, the applied force may be typically between 5-10 Newtons, commonly around 5.5, 6, 7, 8, or 9 Newtons, though it can sometimes exceed 10 Newtons (up to 50 Newtons in some implementations).

In an embodiment, the amount of force applied by the mechanism is calibrated so that the hip capsule ligaments are stretched to approximately where they transition to or begin exhibiting hardened behavior. This ensures optimal tension where the ligaments are appropriately taut without excessive strain that could lead to tissue damage.

Femoral head offset tool 200 automatically finds the optimal position and it applies a consistent, calibrated force that stretches the ligaments to their ideal tension point, just before they enter the hardened behavior region, thereby achieving balanced soft tissue tension throughout the hip joint's range of motion.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments).

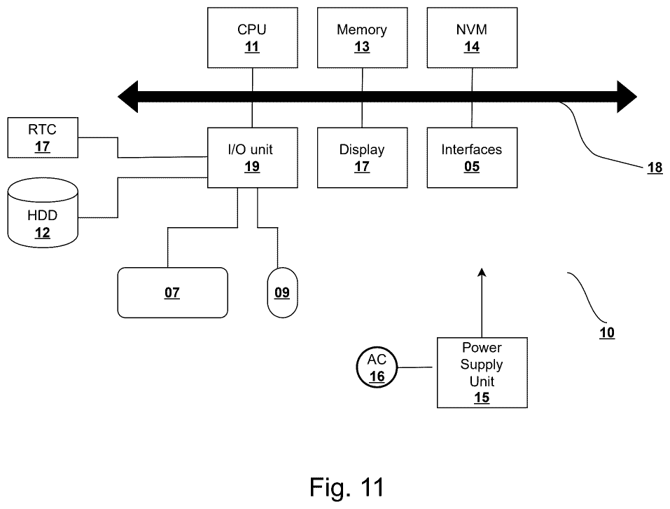

Referring now to FIG. 11, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be adapted to communicate with a plurality of other computing devices, such as clients or servers, over communications networks such as a wide area network, a metropolitan area network, a local area network, a wireless network, the Internet, or any other network, using known protocols for such communication, whether wireless or wired.

CPU 11 is connected to bus 18, memory 13, non-volatile memory (NVM) 14, display 17, I/O unit 19, and Interfaces 5. I/O unit 19 may, typically, be connected to keyboard 09, pointing device 09, hard disk 12, and real-time clock (RTC) 17. Interfaces 05 are designed to connect to a network, which may be the Internet or a local network, which local network may or may not have connections to the Internet. Also shown as part of computing device 10 is power supply unit 15 connected, in this example, to ac supply 16.

I/O unit 19 may include input and out devices. Input devices may be of any type suitable for receiving user input, including for example a keyboard, touchscreen, microphone (for example, for voice input), mouse, touchpad, trackball, or any combination thereof. Output devices may be of any type suitable for providing output to one or more users and may include for example one or more screens for visual output, speakers, printers, or any combination thereof.

Memory 13 may be random-access memory having any structure and architecture known in the art, for use by processors, for example to run software. In a specific embodiment, memory 13 (such as non-volatile random-access memory (RAM) and/or read-only memory (ROM), including for example one or more levels of cached memory) may also form part of CPU 11. However, there are many different ways in which memory 13 may be coupled to computing device 10. Memory 13 may be used for a variety of purposes such as, for example, caching and/or storing data, programming instructions, and the like.

In one embodiment, computing device 10 includes one or more central processing units (CPU) 11, one or more interfaces 05, and one or more bus 18 (such as a peripheral component interconnect (PCI) bus). When acting under the control of appropriate software or firmware, CPU 11 may be responsible for implementing specific functions associated with the functions of a specifically configured computing device or machine. In at least one embodiment, CPU 11 may be caused to perform one or more of the different types of functions and/or operations under the control of software modules or components, which for example, may include an operating system and any appropriate applications software, drivers, and the like.

CPU 11 may include one or more processors such as, for example, a processor from one of the Intel, ARM, Qualcomm, and AMD families of microprocessors. In some embodiments, processors may include specially designed hardware such as application-specific integrated circuits (ASICs), electrically erasable programmable read-only memories (EEPROMs), field-programmable gate arrays (FPGAs), and so forth, for controlling operations of computing device 10. It should be further appreciated that CPU 11 may be one of a variety of system-on-a-chip (SOC) type hardware that may include additional hardware such as memory or graphics processing chips, such as a Qualcomm SNAPDRAGON™ or Samsung EXYNOS™ CPU or AMD Ryzen™ processor or Intel Xeon™ processor or others as are becoming increasingly common in the art, such as for use in mobile devices or integrated devices. As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit. Processors may carry out computing instructions under control of an operating system such as, for example, a version of Microsoft's WINDOWS™ operating system, Apple's Mac OS/X or iOS operating systems, some variety of the Linux operating system, Google's ANDROID™ operating system, or the like stored in memory.

In one embodiment, interfaces 05 enable wired or wireless communication between computing device 10 and another device via a network. Interfaces 05 are provided as network interface cards (NICs). Generally, NICs control the sending and receiving of data packets over a computer network; other types of interfaces 05 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (Wi-Fi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 05 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 11 illustrates one specific architecture for a computing device 10 for implementing one or more of the inventions described herein, it is by no means the only device architecture on which at least a portion of the features and techniques described herein may be implemented. For example, architectures having one or any number of processors may be used, and such processors may be present in a single device or distributed among any number of devices. In one embodiment, a single processor handles communications as well as routing computations, while in other embodiments a separate dedicated communications processor may be provided. In various embodiments, different types of features or functionalities may be implemented in a system according to the invention that includes a client device (such as a tablet device or smartphone running client software) and server systems (such as a server system described in more detail below).

Regardless of network device configuration, the computing device the present invention may employ one or more memories or memory modules (such as, for example, remote memory block and local memory) configured to store data, program instructions for the general-purpose network operations, or other information relating to the functionality of the embodiments described herein (or any combinations of the above). Program instructions may control execution of or comprise an operating system and/or one or more applications, for example. Memory 13 may also be configured to store operating systems, data structures, configuration data, encryption data, historical system operations information, or any other specific or generic non-program information described herein. Because such information and program instructions may be employed to implement one or more systems or methods described herein, at least some network device embodiments may include non-transitory machine-readable storage media, which, for example, may be configured or designed to store program instructions, state information, and the like for performing various operations described herein. Examples of such non-transitory machine-readable storage media include, but are not limited to, magnetic media such as hard disks 12, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a Java™ compiler and may be executed using a Java virtual machine or equivalent, or files containing higher level code that may be executed by the computer using an interpreter (for example, scripts written in Python, Perl, Ruby, Groovy, or any other scripting language).

Computing device 10 includes processors that may run software that carry out one or more functions or applications of embodiments of the invention, such as for example a client application. In many cases, one or more shared services may be operable in computing device 10, and may be useful for providing common services to client applications. Services may for example be WINDOWS™ services, user-space common services in a Linux environment, or any other type of common service architecture used with operating system.

Not shown are batteries that could be present, and many other devices and modifications that are well known but are not applicable to the specific novel functions of the current system and method disclosed herein. It should be appreciated that some or all components illustrated may be combined, such as in various integrated applications (for example, Qualcomm or Samsung SOC-based devices), or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

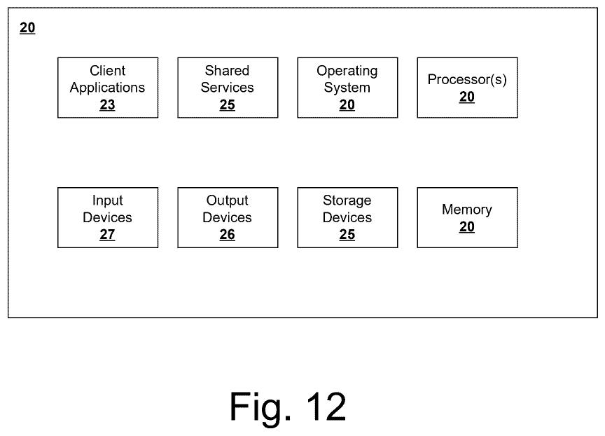

In some embodiments, systems of the present invention may be implemented on a distributed computing network, such as one having any number of clients and/or servers. Referring now to FIG. 12, there is shown a block diagram depicting an exemplary architecture 20 for implementing at least a portion of a system according to an embodiment of the invention on a distributed computing network. According to the embodiment, any number of clients 23 may be provided. Each client 23 may run software for implementing client-side portions of the present invention; clients may comprise a system 20 such as that illustrated in FIG. B. In addition, any number of servers 24 may be provided for handling requests received from one or more clients 23. Clients 23 and servers 24 may communicate with one another via one or more electronic networks 21, which may be in various embodiments any of the Internet, a wide area network, a mobile telephony network (such as CDMA or GSM cellular networks), a wireless network (such as Wi-Fi, WiMAX, LTE, and so forth), or a local area network (or indeed any network topology known in the art; the invention does not prefer any one network topology over any other). Networks 21 may be implemented using any known network protocols, including for example wired and/or wireless protocols.

In addition, in some embodiments, servers 24 may call external services 27 when needed to obtain additional information, or to refer to additional data concerning a particular call. Communications with external services 27 may take place, for example, via one or more networks 21. In various embodiments, external services 27 may comprise web-enabled services or functionality related to or installed on the hardware device itself. For example, in an embodiment where client applications are implemented on a smartphone or other electronic device, client applications may obtain information stored in a server system 24 in the cloud or on an external service 27 deployed on one or more of a particular enterprises or user's premises.

In some embodiments of the invention, clients 23 or servers 24 (or both) may make use of one or more specialized services or appliances that may be deployed locally or remotely across one or more networks 21. For example, one or more databases 25 may be used or referred to by one or more embodiments of the invention. It should be understood by one having ordinary skill in the art that databases 25 may be arranged in a wide variety of architectures and using a wide variety of data access and manipulation means. For example, in various embodiments one or more databases 25 may comprise a relational database system using a structured query language (SQL), while others may comprise an alternative data storage technology such as those referred to in the art as “NoSQL” (for example, Hadoop Cassandra, Google BigTable, and so forth). In some embodiments, variant database architectures such as column-oriented databases, in-memory databases, clustered databases, distributed databases, or even flat file data repositories may be used according to the invention. It will be appreciated by one having ordinary skill in the art that any combination of known or future database technologies may be used as appropriate, unless a specific database technology or a specific arrangement of components is specified for a particular embodiment herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, most embodiments of the invention may make use of one or more security systems 28 and configuration systems 26. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments of the invention without limitation, unless a specific security 28 or configuration system 26 or approach is specifically required by the description of any specific embodiment.

In various embodiments, functionality for implementing systems or methods of the present invention may be distributed among any number of client and/or server components. For example, various software modules may be implemented for performing various functions in connection with the present invention, and such modules may be variously implemented to run on server and/or client components.

The skilled person will be aware of a range of possible modifications of the various embodiments described above. Accordingly, the present invention is defined by the claims and their equivalents.

What is claimed is:

1. A hip joint balancing system, the system comprising:
a femoral head offset tool positioned adjacent to femoral stem and interfaces with an acetabular structure, wherein the femoral head offset tool comprises:
a head including a chamber extending linearly from an opening to a closed end, wherein the head is configured to interface with the acetabular structure, wherein the acetabular structure is elected from a group comprising an acetabular cup, a native acetabulum, and an acetabular defect;
a collar configured to attach to the femoral stem;
force sensors positioned within at least one of the head, the collar, or an interface between the head and collar to measure multi-axis forces transmitted through the femoral head offset tool during a range of motion testing;
an IMU (Inertial Measurement Unit) to measure orientation data, wherein the orientation data is representative of spatial positioning of the femoral head offset tool; and
a spring-loaded ratcheting mechanism comprising a spring system configured to automatically adjust femoral head offset by moving the head relative to the collar by applying spring force against soft tissue resistance during surgeon-guided range of motion testing until force equilibrium is achieved;
a reference module attached to a bone structure to establish orientation reference for measuring relative motion between femur and the bone structure;
a sensing controller comprising a processor and memory and wireless communication interface, the processor configured to:
receive the multi-axis force data from the force sensors and the orientation data from the IMU via the wireless communication interface;
receive the reference orientation data from the reference module;
process the multi-axis force data and the orientation data to monitor an automatic adjustment process of the spring-loaded ratcheting mechanism balancing spring force against soft tissue resistance, wherein the sensing controller provides real-time feedback on a display during range of motion testing and surgeon manipulation;
analyze both the multi-axis force data and the orientation data to detect when spring force equals soft tissue resistance and positional stability is achieved, wherein force equilibrium combined with position stability indicates optimal femoral head positioning; and
responsive to detecting spring force equals soft tissue resistance, provide an indicator that force equilibrium is achieved.

2. The system of claim 1, wherein the spring-loaded ratcheting mechanism in the femoral head offset tool further comprises:
a spring system calibrated to physiological joint tension requirements;
a unidirectional ratchet preventing backward movement of the head relative to the collar; and
a locking mechanism to fix femoral head offset position upon achieving optimal balance, wherein the femoral head offset tool adjusts femoral head offset during the range of motion testing to achieve optimal hip joint tension.

3. The system of claim 2, wherein the locking mechanism comprises:
a locking pin configured to translate through an opening in the head to align with an opening in the collar; and
a plurality of teeth, wherein the locking pin engages with one of the teeth to secure the femoral head offset position.

4. The system of claim 2, wherein the locking mechanism is selected from the group consisting of: ratchet and pawl system, torsion spring system, pin-based system, and external clamp system.

5. The system of claim 1, wherein the reference module comprises:
a clamp assembly configured to attach to a bone structure;
a reference IMU housing containing orientation sensors;
a wireless transmitter configured to communicate orientation data to the sensing controller; and
a battery compartment containing a rechargeable power source.

6. The system of claim 1, wherein determining achievement of force equilibrium comprises analyzing force patterns across multiple axes during a complete range of motion cycle.

7. The system of claim 1, wherein performing range of motion testing comprises moving the hip through flexion-extension motion, abduction-adduction motion, and internal-external rotation.

8. The system of claim 1, wherein the spring system applies force continuously during the range of motion testing while the sensing controller simultaneously monitors force progression to detect equilibrium in real-time.

9. A method for balancing a hip joint during arthroplasty surgery, the method comprising:
positioning a femoral head offset tool between a femoral stem and an acetabular cup, wherein the femoral head offset tool comprises a head configured to interface with the acetabular cup, a collar configured to attach to the femoral stem, force sensors, an IMU, and a spring-loaded ratcheting mechanism comprising a calibrated spring system;
attaching a reference module to a bone structure to establish orientation reference for measuring relative motion between femur and the bone structure;
performing surgeon-guided range of motion testing for:
measuring multi-axis forces between the femoral stem and the acetabular cup;
automatically adjusting femoral head offset by applying spring force against soft tissue resistance until force equilibrium is achieved;
monitoring, by a sensing controller, the automatic adjustment process as the spring force balances against soft tissue resistance;
providing real-time feedback during range of motion testing and surgeon manipulation;
detecting when spring force equals soft tissue resistance indicating optimal positioning; and
providing via equilibrium status indicators, confirmation that force equilibrium is achieved.

10. The method of claim 9, wherein automatically adjusting femoral head offset comprises:
progressively moving head away from a collar automatically to adjust femoral head offset by applying spring force against soft tissue resistance during surgeon-guided range of motion testing until force equilibrium is achieved, wherein the collar remains fixed to the femoral stem; and
preventing backward movement of the head relative to the collar using a unidirectional ratchet mechanism.

11. The method of claim 9, wherein the method further comprises engaging a locking mechanism to secure the femoral head offset position upon achieving optimal balance.

12. The method of claim 9, wherein monitoring the automatic adjustment process further comprises the steps of:
tracking spring force progression in real-time;
measuring soft tissue resistance changes; and
calculating force equilibrium status during range of motion testing.

13. The method of claim 9, wherein providing real-time feedback during range of motion testing and surgeon manipulation comprises a real-time force visualization interface showing spring force and soft tissue resistance, and the equilibrium status indicators to confirm when optimal balance is achieved.

14. The method of claim 9, wherein determination of force equilibrium further comprises analyzing force symmetry across multiple axes during a complete range of motion cycle.

15. The method of claim 9, wherein detecting when spring force equals soft tissue resistance further comprises the steps of:
monitoring force progression in real-time;
identifying when incremental force results in minimal displacement; and
confirming biomechanical equilibrium across multiple axes.

16. The method of claim 9, wherein the method further comprises:
measuring, by the surgeon, using a sizing tool, the femoral head offset distance when equilibrium is achieved; and
selecting a permanent femoral head implant based on the measured offset distance.

17. The method of claim 9, wherein performing range of motion testing comprises moving the hip through flexion-extension motion, abduction-adduction motion, and internal-external rotation.

18. A hip joint balancing tool, comprising:
a head including a chamber extending linearly from an opening to a closed end, wherein the head is configured to interface with an acetabular structure;
a collar configured to attach to a femoral stem;
an IMU positioned within at least one of the head and the collar to measure orientation data changes during the automatic adjustment process;
a spring-loaded mechanism configured to automatically adjust femoral head offset by moving the head relative to the collar until force equilibrium is achieved, wherein the IMU detects when adjustment movement has ceased indicating force equilibrium; and
a locking mechanism to secure the head position relative to the collar at optimal offset.

19. The hip joint balancing tool of claim 18, wherein the acetabular structure is selected from the group consisting of: an acetabular cup, a native acetabulum, or an acetabular defect.

20. The hip joint balancing tool of claim 19, wherein the IMU is positioned within the head to measure orientation changes during acetabular structure interface.

21. The hip joint balancing tool of claim 19, wherein the spring-loaded mechanism comprises at least one of:
a coil spring disposed in the chamber between the collar and the closed end;
magnets with opposing polarities creating repulsive force between the collar and the closed end; or
an electromagnetic actuator configured to move the collar relative to the head; or
a compressed gas mechanism providing controlled force application.

22. The hip joint balancing tool of claim 19, wherein the tool is configured for use during surgeon-guided range of motion testing including flexion-extension, abduction-adduction, and internal-external rotation.

23. The hip joint balancing tool of claim 19, wherein the head and collar comprise biocompatible materials suitable for temporary surgical contact.

24. The hip joint balancing tool of claim 19, wherein force equilibrium is achieved when the spring force balances against soft tissue resistance during range of motion testing.
